(19) United States
(12) Patent Application Publication
(10) Pub. No.: US 2023/0078909 A1
(43) Pub. Date: Mar. 16, 2023

(54) LOCKING MECHANISM FOR A MEDICAMENT DELIVERY DEVICE

(71) Applicant: SHL MEDICAL AG, ZUG (CH)

(72) Inventor: Anders Böstrom, Ingarö (SE)

(21) Appl. No.: 17/798,649

(22) PCT Filed: Dec. 15, 2020
(86) PCT No.: PCT/EP2020/086279

§ 371 Date: Aug. 10, 2022

(30) Foreign Application Priority Data
Mar. 26, 2020 (EP) 20165832.5

(51) Int. Cl. A61M 5/315; A61M 5/32; A61M 5/34

(52) U.S. Cl. CPC A61M 5/31571; A61M 5/3202; A61M 5/344

(57) ABSTRACT

A locking mechanism for use in a medicament delivery device prevents unintended movement or premature activation of a medicament delivery member cover while a protective cap is attached to the medicament delivery device. Removal of the protective cap unlocks the locking mechanism that can include a protrusion projecting radially inward from an interior portion of a protective cap and a locking ring having an annular shape and being rotatably positioned within the interior of the cap. The locking ring may include a first slot and a second slot, wherein the protrusion is slidably positioned within the first slot and the second slot is located at a distal end of the locking ring. A through hole opening in the locking ring allows the locking ring to rotate relative to the medicament delivery member cover from a locked position to an unlocked position when the cap is moved axially.

### TECHNICAL AREA

The present disclosure relates to a locking mechanism for a medicament delivery member cover that is part of a medicament delivery device to prevent unintended movement of the medicament delivery member cover while a protective cap is attached to the medicament delivery device. Removal of the protective cap unlocks the locking mechanism.

### BACKGROUND

Many medicament delivery devices on the market today are arranged with a number of features that will facilitate the use of the device and in particular since the devices more and more are intended to be handled by the patients themselves. The added functionality is also an advantage when the medicament delivery devices comprise injection needles since many patients feel uncomfortable seeing the needle and also feel reluctant regarding penetration of the needle. In that respect, an added functionality such as automatic penetration mechanisms as well as automatic injection mechanisms, to mention some features.

Many medicament delivery devices that are arranged with automatic features and functions are often arranged with some sort of actuation member at the proximal end of the device. These actuation members may be sleeve-like members that are pressed against the dose delivery site whereby they can be moved in the distal direction in relation to the device, thereby activating a certain feature or function. In addition, these sleeve-like members may also function as medicament delivery member guards or covers that hide the medicament delivery member before, during and after dose delivery.

Even though these delivery member guards offer a solution relating to the hiding of a delivery member, e.g., an injection needle, the inclusion of such guards in a medical device can introduce a risk that the medicament delivery device may be accidentally or prematurely activated if the device is jostled during shipment or dropped on a hard surface, for example, if a user accidentally drops the delivery device on the floor. Such an impact may well cause the actuation member, i.e., the guard, at the proximal end of the delivery device to move to such an extent that the delivery device is activated such that a dose of medicament is delivered. This unintended delivered dose of medicament is then wasted. Such a problem is enhanced if the delivery device is designed and configured as a non-reusable device. In those situations, the delivery device then has to be discarded, typically without having delivered any medicament to the patient and a new device has to be obtained. Even if the device is re-usable, it has to be reloaded in order to deliver a new dose. Both obtaining a new device or reloading a device is costly, time-consuming and is stress-causing to a patient.

There is thus a demand for devices having an increased functionality regarding minimizing the risk of activation of medicament delivery devices after mishandling, accidental drops or other types of accidental impacts.

### SUMMARY

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

One goal of the present disclosure is to remedy the drawbacks of the current state-of-the art medicament delivery devices, more specially where a delivery device is prone to a premature, inadvertent or unintended activation. This goal is obtained by inclusion of a locking mechanism as described herein and its incorporation into the design of a medicament delivery device. Preferable embodiments of the present disclosure form the subject of the below-listed claims.

The scope of protection is defined by the claims, to which reference should now be made.

One embodiment provides a locking mechanism for preventing axial movement of a medicament delivery member cover in a medicament delivery device, the locking mechanism comprising: a cap with a longitudinal axis; a locking ring having an annular shape and being rotatably positioned within the interior of the cap; a medicament delivery member cover rotatably positioned within the interior of the locking ring; a first connection between the cap and the locking ring, the first connection comprising a first slot and a first protrusion in the first slot, wherein the first slot is in one of the locking ring and the cap and the first protrusion is attached to the other of the locking ring and the cap; and a second connection between the locking ring and the medicament delivery member cover, the second connection comprising a second slot and a second protrusion arranged in the second slot, wherein the second slot is in one of the locking ring and the medicament delivery member cover, and the second protrusion is attached to the other of the locking ring and the medicament delivery member cover.

Optionally, the first protrusion is attached to the cap, and the first protrusion projects radially inward from an interior portion of the cap towards a longitudinal axis of the cap. Optionally, the first protrusion is attached to the locking ring, and the first protrusion projects radially outward from an outer portion of the locking ring away from a longitudinal axis of the cap.

Optionally, the second slot is in the locking ring, and the second slot is located at a distal end of the locking ring and has an open end. Optionally, the second slot is in the medicament delivery member cover, and the second slot is located at a proximal end of the locking ring and has an open end. Optionally, the second slot extends from the open end to a closed end, and wherein the second protrusion is in the second slot at the closed end.

Optionally, the locking mechanism of any previous claim, wherein the locking ring is configured to rotate relative to the cap from a locked position to an unlocked position when the cap is moved axially relative to the medicament delivery member cover. Optionally, when the locking ring is in the locked position the medicament delivery member cover cannot move axially relative to an outer housing of the medicament delivery device. Optionally, removal of the cap from the locking mechanism rotates the locking ring relative to the cap and thereby causes the second protrusion to move within the second slot. Optionally, causing the second protrusion to move within the second slot results in the protrusion being axially aligned with the open end of the second slot.

Optionally, the first slot comprises a helical cam. Optionally, the helical cam comprises a cam surface against which the protrusion is pushed during use. Optionally, the second slot is an L-shaped cut-out. Optionally, the locking ring extends from a proximal end to a distal end relative to the longitudinal axis, and wherein the second slot is spaced apart from the proximal end and from the distal end in a direction relative to the longitudinal axis. Optionally, the first protrusion comprises a cam follower that is always slidably engaged with a cam surface on the first slot.

Optionally, the cap further comprises a hollow tube that projects distally from the interior portion parallel to the longitudinal axis and extends beyond a circumferential distal end of the cap. Optionally, the tube comprises a first part of a grabber assembly configured to engage a rigid delivery member shield. Optionally, the grabber assembly further comprises a second part comprising at least two distally extending fingers, each terminating in a shield hook. Optionally, the first slot and the second slot both extend the same distance around the longitudinal axis.

In one embodiment, a medicament delivery device is provided comprising a locking mechanism as described above.

In another possible embodiment of the present disclosure there is presented a locking mechanism for preventing axial movement of a medicament delivery member cover or guard that is part of a medicament delivery device, where the locking mechanism includes a cap having a protrusion projecting radially inward from an interior portion of the cap towards a longitudinal axis of the cap. A locking ring having an annular shape is rotatably positioned within the interior of the cap, wherein the locking ring further has a first slot and a second slot, wherein the protrusion in the cap is slidably positioned within the first slot. A benefit of this engagement of the protrusion with the first slot is that it can allow the locking ring to be removed with the cap when the cap is removed from the delivery device (if desired).

The second slot is located at a distal end of the locking ring and has an open end. The locking ring can have a through hole opening having a diameter large enough to accept a proximal end of a medicament delivery member cover or guard of a medical delivery device such that the locking ring will rotate relative to the medicament delivery member cover. Typically, the guard or cover will be rotationally fixed to the delivery device outer housing that surrounds a dose delivery mechanism. Such a dose delivery mechanism can include a biased plunger rod operatively connected to medicament container having an attached delivery member, such as an injection needle. The locking ring can also be configured to rotate relative to the cap from a locked position to an unlocked position when the cap is moved axially relative to the medicament delivery member cover.

When the locking ring is in the locked position, the medicament delivery member cover preferably cannot move axially relative to an outer housing of the medicament delivery device. A benefit of this prevented axial movement of the cover or guard in a distal direction relative to the outside housing of the delivery device is that it can work to prevent premature or inadvertent activation of those delivery device designs that require axial movement of a cover or guard to either activate or actually fire, i.e., initiate delivery of medicament, the device. The first slot in the locking ring can have a helical shape, such as a helical cam, and the second slot can be configured as an L-shaped cut-out. The protrusion that can be fixed on the interior wall of the cap can be configured as a cam follower that is always slidably engaged with a cam surface on the first slot. Regarding the second slot, the open end is preferably located at a terminal distal end of the locking ring, where that open end has a shape and size to accept a nib located on an outside surface of the medicament delivery member cover or guard. In preferred embodiments the locking ring would include two or more first and second slots that are operatively engaged with two of more corresponding protrusions and nibs. Ideally, a preferred design would use two protrusions and two nibs each of which are respectively separated by 180 degrees on the interior of the cap and outer surface of the cover, respectively. In such a configuration the two first slots and the two second slots are likewise separated from each other circumferentially around the locking ring by 180 degrees such that they can align with the two sets of protrusions and nibs.

The cap of the locking mechanism can be designed as a protective cap with a hollow tube that projects distally from the interior portion parallel to the longitudinal axis and extends beyond a circumferential distal end of the cap. This tube can also have a first part of a grabber assembly configured to engage a rigid delivery member shield, where the grabber assembly further can have a second part comprising at least two distally extending fingers, each terminating in a shield hook. The first and second parts can be separate and distinct components that are connected together, or they can be formed during manufacture of the cap as integral features of the cap, i.e., not as separately manufacture components.

Yet a further embodiment of the present disclosure is directed to a medicament delivery device that includes one of more of the above-described embodiments of the locking mechanism. For example, such a medicament delivery device has a locking mechanism as described above and includes a medicament delivery member cover having a protrusion configured to engage with the second slot of the locking mechanism. Another such embodiment of a medicament delivery device includes a distal portion having an outer housing containing a medicament delivery member cover as part of a dose deliver mechanism, where the delivery member cover or guard protrudes beyond a terminal proximal end of the outer housing prior to activation of the medicament device. One of the possible locking mechanisms of present disclosure can be included for preventing axial movement of the medicament delivery member cover prior to activation, where one possible locking mechanism includes a cap having an interior surface, wherein the interior surface has a protrusion projecting radially inward towards a longitudinal axis of the cap. A locking ring can be included having an annular shape and being rotatably positioned inside the cap, wherein the locking ring further includes a first slot and a second slot, wherein the protrusion is slidably positioned within the first slot and the second slot is located at a proximal end of the ring and has an open end sized to accept a nib located on a proximal end of the medicament delivery member cover. A through hole opening in the locking ring can be present having a diameter large enough to accept the proximal end of the medicament delivery member cover such that the locking ring will rotate relative to the medicament delivery member cover. Preferably, the locking ring is configured to rotate relative to the cap from a locked position to an unlocked position when the cap is moved axially relative to the proximal end of the outer housing.

In another embodiment, a medicament delivery device similar to the above-described device can be configured where the first slot is helical and is always engaged with the protrusion such that axial movement of the cap in the proximal direction relative to the outer housing causes the protrusion to slidably engage with an inside surface of the first slot, wherein movement of the protrusion relative to the inside surface causes the locking ring to rotate relative to the cap. Further, a medicament delivery device could also be designed where the second slot is L-shaped having a first leg that is parallel to the longitudinal axis and terminates at the open end. Preferably, such a device is configured where rotation of locking ring from the locked position to the unlocked position causes a second leg of the second slot to move transversely relative to the nib until the nib is positioned within the first leg directly opposite the open end.

In yet another possible embodiment, the medicament delivery device can be configured where axial movement of the cap out and away from the delivery device housing, when the locking ring is in the unlocked position, will cause the open end to move out of engagement with the nib such that the cap can be completely removed from the outer housing. The locking ring, because of the engagement of the protrusion with the first slot, will also be removed with the cap.

The medicament delivery device can further include a delivery member connected to a proximal end of a medicament container positioned within the outer housing, a rigid delivery member shield surrounding and covering a distal end of the delivery member, and a shield remover, wherein the distal portion further comprises a delivery device connector configured to releasably engage a corresponding connector located on the outer housing. The shield remover could have a hollow tube that projects distally from the interior surface parallel to the longitudinal axis and extends beyond a circumferential distal end of the cap. A grabber assembly can be configured to engage the rigid delivery member shield and the hollow tube could be design as a first part of a grabber assembly configured to engage the rigid delivery member shield. The grabber assembly further could have a second part having at least two distally extending fingers each terminating in a shield hook, where the shield hooks can engage the rigid delivery member shield when the cap is moved in a distal direction relative to the outer housing causing the rigid delivery member shield to disengage from the delivery member.

The medicament delivery device according to the present disclosure may also preferably comprise a generally elongated housing, having a proximal and a distal end. The device is preferably further arranged with a medicament delivery actuation mechanism. This mechanism may be designed in many ways. A preferable solution is to utilize an elongated plunger rod that is capable of acting on a sliding stopper of a medicament container such that proximal movement of the plunger rod relative to the delivery device outer housing causes axial movement of the stopper which in turn exerts pressure on medicament in a container to cause the medicament to flow out of an attached delivery member, e.g., an injection needle.

The plunger rod may be arranged to be moved purely manually or by some sort of force. In this respect, resilient force members such as springs may be used for creating the necessary axially directed force. The force may then be acting directly on the plunger rod or via drive mechanisms operatively engaged with the plunger rod, such as threaded nuts cooperating with threads on the plunger rod. Further, there may be mechanisms that will activate the force to perform dose delivery. These mechanisms may comprise contact members that will activate the device when the contact member is pressed against a dose delivery site. In this respect the contact member may be a medicament delivery member guard, shield or cover as indicated above arranged movable relative to the proximal end of the housing.

The movable delivery member cover or guard will thus surround the delivery member which is an advantage if the person using the device is afraid of medicament delivery members and in particular injection needles. It is also to be understood that the device may further comprise additional functions such as penetration mechanisms for injection needles, which also may be activated by the contact member(s).

In order to prevent damage to the proximal end of the delivery device and to protect the medicament delivery member before use, the previously mentioned protective cap may be releasably arranged to the proximal end of the housing. The protective cap may preferably include a medicament delivery member shield remover for removing protective members that surround a medicament delivery member before use. It may for example be injection needle sheaths that surround injection needles, keeping them sterile before use, such as rigid needle shields or flexible needle shields.

The advantage of including the locking mechanisms embodied by the present disclosure in medicament delivery device designs is that they ensure that the medicament delivery member guard, shield or cover is not affected if someone accidentally drops a device onto a surface such as a floor or a table or any other hard surface. Likewise, the locking mechanism can protect against other rough device handling, for example, during ship of the device. The device will thus not be inadvertently triggered, or components and/or functions will not be moved and/or activated due to accidental improper handling. The locking mechanisms of the present disclosure will thus ensure that the device will stay in operational mode even if the device is not correctly handled. In addition, the cooperation between the medicament delivery member shield remover of the protective cap provides a robust solution that enables an unlocking of the medicament delivery member guard when the protective cap is removed. Thus, the medicament delivery member guard is locked as long as the protective cap with its medicament delivery member shield remover is attached to the medicament delivery device.

According to another possible favorable solution the medicament delivery member shield remover may comprise inclined tongues arranged to grip into an outer surface of the medicament delivery member shield. This provides a firm and positive gripping of the medicament delivery member shield by the medicament delivery member shield remover. In order to provide a very secure and firm gripping of the medicament delivery member shield, the medicament delivery member shield remover may be made of metal. Additionally, the protective cap may include a protective cap holding mechanism, operably arranged to releasably hold the protective cap. One such protective cap holding mechanism may include a protrusion on a housing part of the delivery device, forming contact surfaces, which contact surfaces are arranged to cooperate with contact surfaces on the protective cap. With this solution, the protective cap is held in place on the housing until a user applies a pulling or twisting force on the protective cap for removal before use.

The protective cap may be designed of a material with resilient properties and the contact surfaces may be arranged such that a release force on said protective cap will cause the protective cap to flex and said contact surfaces to be moved out of contact with each other, thereby releasing the protective cap. With the solution, the material properties of the protective cap are utilized for the holding function.

These and other aspects of, and advantages with, the present disclosure will become apparent from the following detailed description of the disclosure and from the accompanying drawings.

### BRIEF DESCRIPTION OF DRAWINGS

In the following detailed description of the invention, reference will be made to the accompanying drawings, of which

FIG. 1 shows a perspective view of one possible medicament delivery device design that can incorporate one embodiment of the locking mechanism of the present disclosure;

FIG. 2 is an exploded view of the delivery device of FIG. 1;

FIG. 3A is transparent view of one possible design of the locking mechanism of the present disclosure in a locked configuration;

FIG. 3B is transparent view of the locking mechanism of FIG. 3A in an unlocked configuration;

FIG. 4A is a perspective view of one possible design of the locking ring of the present disclosure;

FIG. 4B is a perspective view of one possible design of the medicament delivery guard of the present disclosure;

FIG. 4C is a perspective view of the locking ring of FIG. 4A engaged with the medicament delivery guard of FIG. 4B;

FIG. 5A is a perspective view of one possible design of the protective cap of the present disclosure;

FIG. 5B is a perspective view of the protective cap of FIG. 5A engaged with the locking ring of FIG. 4A; and

FIG. 6 shows part of another locking mechanism design.

FIG. 7 shows the design of FIG. 6 during use of the locking mechanism.

FIG. 8 shows a cross-section view of part of the locking mechanism design of FIG. 6.

FIG. 9 shows a perspective view of the part of the locking mechanism design of FIG. 6.

### DETAILED DESCRIPTION

A number of embodiments of the invention will now be described with the help of the figures. The figures show examples of a locking mechanism with the second slot in the locking ring and a locking mechanism with the second slot in the medicament delivery member cover. A number of other alternatives are also described in the specification.

In general, a locking mechanism for preventing axial movement of a medicament delivery member cover 54 in a medicament delivery device will now be described. The locking mechanism comprises a cap 42 with a longitudinal axis, a locking ring 60 having an annular shape and being rotatably positioned within the interior of the cap 42, and a medicament delivery member cover 54 rotatably positioned within the interior of the locking ring 60. There is a first connection between the cap 42 and the locking ring. The first connection comprises a first slot 61 and a first protrusion 70 in the first slot 61. In general, the first slot 61 is in one of the locking ring 60 and the cap 42 and the first protrusion 70 is attached to the other of the locking ring 60 and the cap 42. There is a second connection between the locking ring 60 and the medicament delivery member cover 54. The second connection comprises a second slot 62 and a second protrusion 55 arranged in the second slot 62. In general, the second slot 62 is in one of the locking ring 60 and the medicament delivery member cover 54, and the second protrusion 55 is attached to the other of the locking ring 60 and the medicament delivery member cover 54.

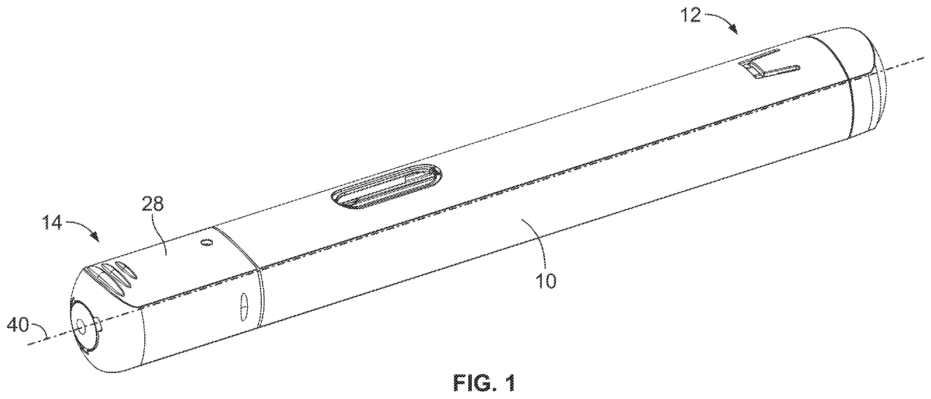

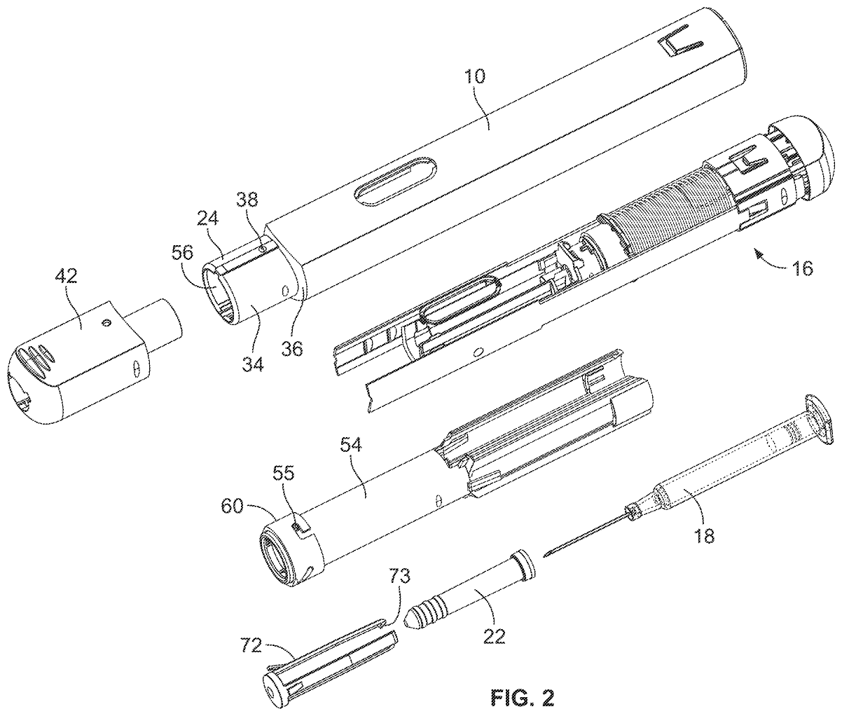

The embodiment of the medicament delivery device of the present disclosure shown in FIGS. 1 to 5B is designed with a generally elongated, tubular housing 10 that can be generally round or somewhat triangular in cross-sectional shape and having a distal end 12 and a proximal end 14, FIG. 1. The housing 10 is arranged to accommodate a medicament delivery actuation mechanism 16, FIG. 2, capable of, upon activation, effecting a delivery of a dose of medicament from a medicament container 18 arranged inside the housing via a medicament delivery member, such as an injection needle, which is shown protected and covered by delivery member shield 22. The medicament delivery member shield 22, as shown, is a so called RNS (rigid needle shield). This shield 22 is removed from medicament container 18 by a medicament delivery member shield remover 72, which is axially fixed to protective cap 42. The medicament delivery actuation mechanism may have a number of designs and functions that are applicable and functional with the locking mechanisms disclosed in the present disclosure.

The proximal end of the housing 10 has a terminal end 24 having a reduced diameter and a generally tubular shape. Although shown as an integrated portion of housing 10, e.g., fabricated a single moulded piece, the terminal end 24 could also be configured and designed as a separate connectable part. When the design includes two separate outer housing components, the distal part of the terminal 24 can be arranged with dimensions and a shape generally corresponding to a proximally directed opening of the housing 10 such that the distal part can be arranged with attachment members engaged with corresponding attachment members, like a permanent snap-fit connector, on an inside surface of the housing.

The terminal end 24 further can have a proximal part 34 with a somewhat lesser diameter than the diameter of housing 10. The proximal part 34 of the terminal end 24 is separated from the distal portion of the housing 10 by a generally proximally directed transition surface 36. Further, the terminal end 24 is arranged with a first holding member 38 that can form part of a protective cap holding mechanism on the outer surface of the proximal part 34. One example of the holding member 38 is shown as a ledge or protrusion positioned generally in line with the longitudinal direction 40 of the device, FIG. 1. The protrusion 38 may have somewhat inclined surfaces to assist in connecting and removing of the protective cap 42.

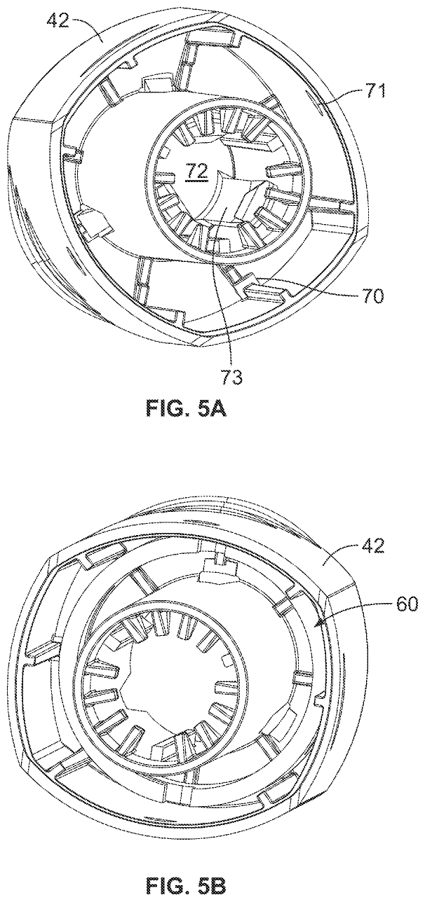

The protective cap 42, FIG. 2, is designed to engaged and form a removable connection with proximal part 38 so that the cap 42 becomes attached to and in abutment with housing 10. The protective cap 42, like the housing 10, is generally tubular in shape and has a distally directed opening 44, FIG. 5A, which has a diameter somewhat larger than the outer diameter of the proximal part 34 of the terminal end 24, such that the protective cap 42 will fit around the proximal part 34. On the inner surface of the protective cap 42 a second holding member 71 is arranged, designed to interact with the first holding member 38. Together these features define a protective cap holding mechanism. In the embodiment shown, holding member 38 has a protrusion 39 that is configured to cooperate and engage with second holding member 71, which can be design as an indent to accept and hold protrusion 39.

The protective cap 42 is further arranged with a medicament delivery member shield remover 72, shown in the embodiment in the form of a tubular needle shield remover 72 attached to a distally directed wall surface of cap 42. The needle shield remover 72 is arranged with inwardly extending inclined tongues 73, which are capable of gripping into the outer surface of the RNS 22, FIG. 2.

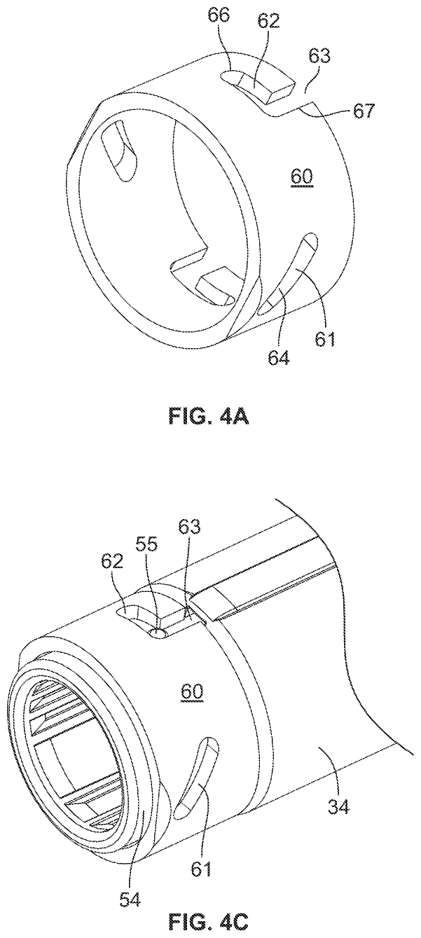

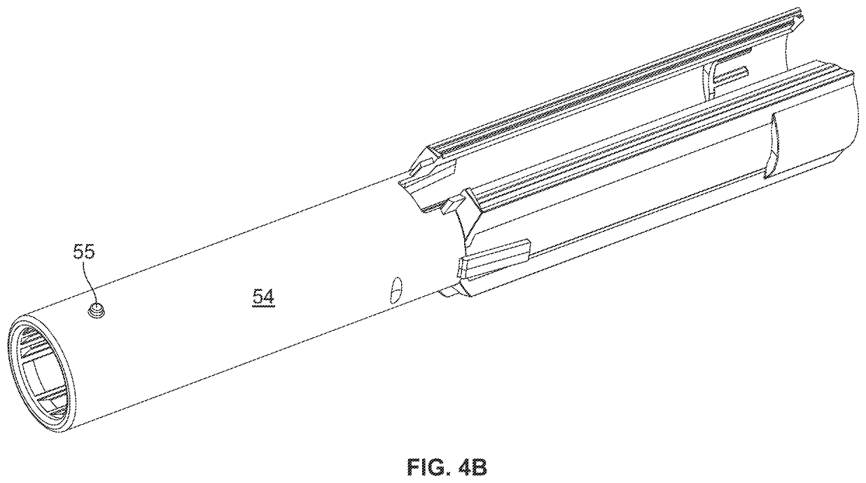

The device further comprises a medicament delivery member cover or guard 54, FIGS. 2 and 4B, in the form of a generally elongated tubular member where the proximal part has a diameter only somewhat smaller than the inner diameter of an opening 56 at the proximal end of the terminal end 24. The medicament delivery member cover 54 is arranged slidable in the longitudinal direction of the device from positions where the medicament delivery member is covered to positions where the medicament delivery member is exposed during delivery of medicament. An actuator may be arranged to force the medicament delivery member guard 54 in the proximal direction.

The outer surface of the medicament delivery member guard 54 is arranged with a nib 55, FIGS. 2 & 4B. In the embodiment shown, the nib is position at the proximal end of the cover 54 and projects radially outward from the longitudinal axis 40. This nib 55 is designed and configured with a shape such that is fit within and be slidably retained in the second slot 63. Opening 63 in the second slot 62 is sized and configured to slidably receive nib 55, FIG. 4C.

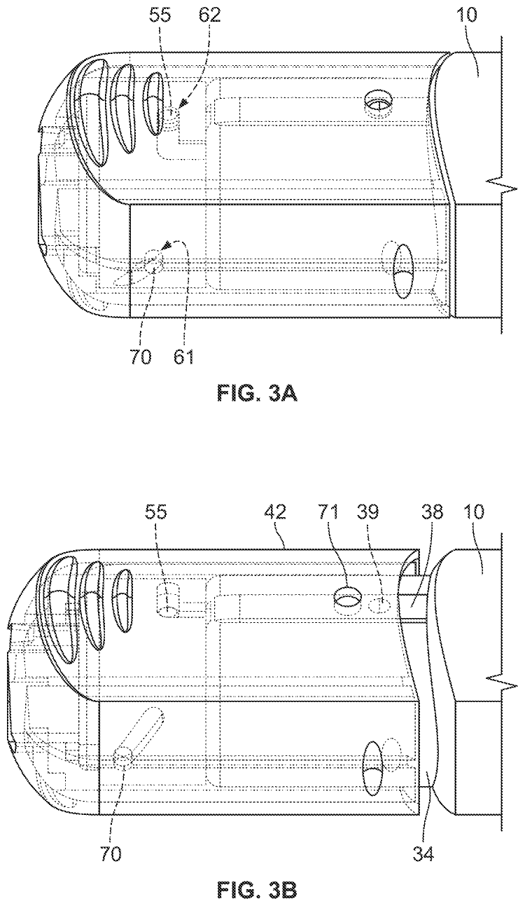

When the device is in an initial state with the medicament delivery member guard or cover 54 in an initial position, FIG. 3A, the locking mechanism (combination of 55, 60 & 70) is in a locked configuration. FIG. 3B shows the locking mechanism in the unlocked configuration. The cap 42 in the embodiment shown is designed to be pulled axially off housing end cap 24, not rotated off. As such, the axial movement of the cap 42 in the proximal direction causes the protrusion 70, designed as a cam follower, to also move axially since it is connected to an inside surface of the cap. During use, as protrusion 70 moves axially with the cap 42, the protrusion 70 rides against a cam surface 64 of the first slot 61, which forces the locking ring 60 to rotate relative to the medicament delivery member cover 54, which is rotationally fixed relative to housing 10. The rotation of the locking ring 60 simultaneously causes movement of second slot 62 relative to the nib 55 on the medicament delivery member cover 54. The second slot 62 is shown configured as L-shaped having a first leg 67 that is parallel to the longitudinal axis and terminates at the open end 63 and a second leg 66 that is generally transverse to the first leg. The second slot 62 moves relative to the stationary nib 55, i.e., rotates because the locking ring 60 is rotating, until it eventually aligns with the open end 63 of the second slot 62, FIG. 3B. Further axial movement of the cap 42 and protrusion 70 will result in the open end 63 of the second slot 62 disengaging from the nib 55 to allow the cap 42 to be completely removed. The inclined tongues 73 of needle shield remover 72 will engage the shield 22 as the cap 42 is pulled axially resulting in the shield 22 being removed from the proximal end of the medicament container and exposing the injection needle.

Once the cap 42 and locking ring 60 are removed from the guard 54, the guard can now move axially in a distal direction to activate the medicament delivery device, for example, by pressing the guard 54 against an injection site. The pressing of the medicament delivery guard 54 will cause it to move in the distal direction in relation to the housing and the terminal end 24. Thus, the distal movement of the medicament delivery member guard 54 in the distal direction will cause an activation of the device for delivering a dose of medicament.

In the locked configuration of the locking mechanism, as shown in FIG. 3A, the nib 55 is positioned in the second leg 66 of the second slot 62. Axial movement of the nib 55, and as such the guard 54, thus prevented. Preventing axial distal movement guard 54 will prevent unintended activation of the delivery device. As stated, cap removal causes rotation of locking ring 60 from a locked position to an unlocked position and causes the second leg 66 of the second slot 62 to move transversely relative to the nib 55 until the nib is positioned within the first leg 67 directly opposite the open end 63. In this unlocked locking ring position, the nib 55 and the locking ring can be disengaged from each other.

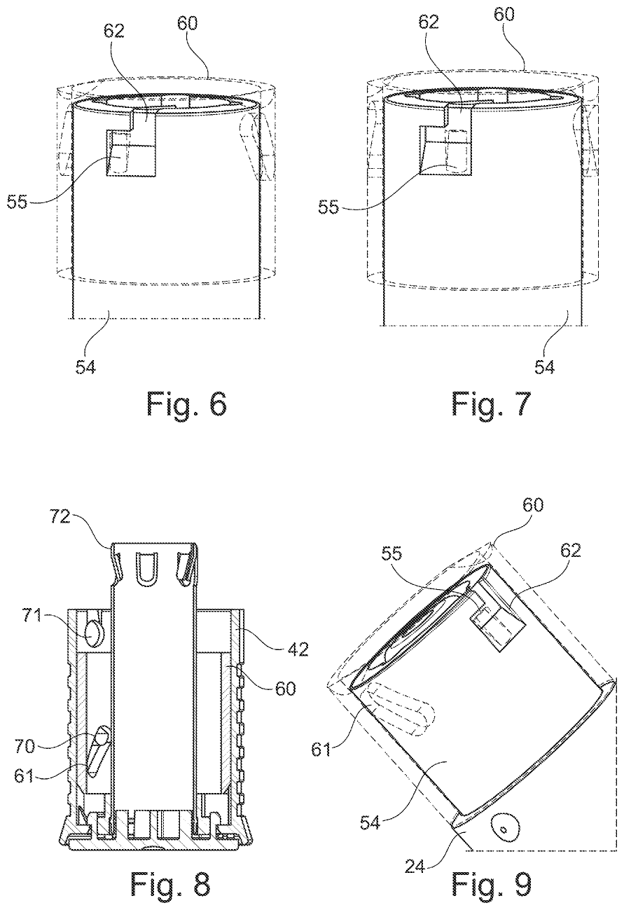

FIGS. 6 to 9 show another embodiment. Whilst various minor features are different in this embodiment, perhaps the most substantial difference is in the second connection, where the nib (or protrusion) 55 is attached to the locking ring 60 and the second slot 62 is in the guard 54. FIGS. 6 and 9 show the relative positions of the locking ring 60 and the guard 54 before use, when the nib 55 is not aligned with the open end of the second slot 62 in the axial direction. FIG. 7 shows the relative positions of the locking ring 60 and the guard 54 during use, where the locking ring 60 and the guard 54 have moved relative to one another in the rotational direction, meaning that the nib 55 is now aligned with the open end of the second slot 62. FIG. 8 shows the position of the cap 42 relative to the locking ring 60 before use. An alternative medicament delivery member shield remover 72 is also shown here, although the medicament delivery member shield remover 72 shown in FIG. 2 could also be used.

Many alternatives to the embodiments described above are also possible. One example of a medicament delivery device in which the locking mechanism could be used is provided in the figures, but other medicament delivery devices could also incorporate the locking mechanism described herein. The medicament delivery device may be an autoinjector or a pen injector, for example. Many of the features can also take various alternative shapes—for example, the first slot 61 is shown as a straight slot and the second slot 62 is shown as an L-shaped slot, but these precise shapes are just an example and variations on these shapes are possible. The precise locations of features are also not necessarily fixed, and some features are also optional—for example, the second holding member 71 and the first holding member 38 could be moved to a different location, changed to a different shape, replaced by a different holding system or even removed entirely. The slot 61 could be on the cap 42 rather than on the locking ring 60, with the protrusion 70 on the locking ring 60 rather than on the cap 42. More generally, it is to be understood that the embodiment described above and shown in the drawings is to be regarded only as a non-limiting example of the disclosure and that it may be modified in many ways within the scope of the patent claims.

Various aspects of the invention are summarised in the following clauses.
1. A locking mechanism for preventing axial movement of a medicament delivery member cover in a medicament delivery device, the locking mechanism comprising:

a cap comprising a protrusion projecting radially inward from an interior portion of the cap towards a longitudinal axis of the cap; and

a locking ring having an annular shape and being rotatably positioned within the interior of the cap, wherein the locking ring further comprises,
a first slot;
a second slot, wherein the protrusion is slidably positioned within the first slot and the second slot is located at a distal end of the locking ring and has an open end; and
a through hole opening having a diameter large enough to accept a proximal end of a medicament delivery member cover of a medical delivery device such that the locking ring will rotate relative to the medicament delivery member cover,

wherein the locking ring is configured to rotate relative to the cap from a locked position to an unlocked position when the cap is moved axially relative to the medicament delivery member cover.
2. The locking mechanism of clause 1, wherein when the locking ring is in the locked position the medicament delivery member cover cannot move axially relative to an outer housing of the medicament delivery device.
3. The locking mechanism of clause 1 or 2, wherein the first slot comprises a helical cam.
4. The locking mechanism of any one of clauses 1 to 3, where the second slot is an L-shaped cut-out.
5. The locking mechanism of any one of clauses 1 to 4 where the protrusion comprises a cam follower that is always slidably engaged with a cam surface on the first slot.
6. The locking mechanism of any one of clauses 1 to 5, wherein the open end of the second slot is located at a terminal distal end of the locking ring.
7. The locking mechanism of any one of clauses 1 to 6, where the open end has a shape and size to accept a nib located on an outside surface of the medicament delivery member cover.
8. The locking mechanism of any one of clauses 1 to 7, wherein the cap further comprises a hollow tube that projects distally from the interior portion parallel to the longitudinal axis and extends beyond a circumferential distal end of the cap.
9. The locking mechanism of clause 8, wherein the tube comprises a first part of a grabber assembly configured to engage a rigid delivery member shield.
10. The locking mechanism of clause 9, where the grabber assembly further comprises a second part comprising at least two distally extending fingers, each terminating in a shield hook.
11. A medicament delivery device comprising:

a distal portion comprising an outer housing containing a medicament delivery member cover that protrudes beyond a terminal proximal end of the outer housing prior to activation of the medicament device;

a locking mechanism for preventing axial movement of the medicament delivery member cover prior to activation comprising,
a cap comprising an interior surface, wherein the interior surface has a protrusion projecting radially inward towards a longitudinal axis of the cap; and
a locking ring having an annular shape and being rotatably positioned inside the cap, wherein the locking ring further comprises,
   a first slot;
   a second slot, wherein the protrusion is slidably positioned within the first slot and the second slot is located at a distal end of the locking ring and has an open end sized to accept a nib located on a proximal end of the medicament delivery member cover; and
   a through hole opening having a diameter large enough to accept the proximal end of the medicament delivery member cover such that the locking ring will rotate relative to the medicament delivery member cover,

wherein the locking ring is configured to rotate relative to the cap from a locked position to an unlocked position when the cap is moved axially relative to the proximal end of the outer housing.
12. The medicament delivery device of clause 12, wherein the first slot is helical and is always engaged with the protrusion such that axial movement of the cap in the proximal direction relative to the outer housing causes the protrusion to slidably engage with an inside surface of the first slot, wherein movement of the protrusion relative to the inside surface causes the locking ring to rotate relative to the cap.
13. The medicament delivery device of clause 11 or 12, wherein the second slot is L-shaped having a first leg that is parallel to the longitudinal axis and terminates at the open end.
14. The medicament delivery device of clause 13, where rotation of locking ring from the locked position to the unlocked position causes a second leg of the second slot to move transversely relative to the nib until the nib is positioned within the first leg directly opposite the open end.
15. The medicament delivery device of any of clauses 12-14, where axial movement of the cap when the locking ring is in the unlocked position will cause the open end to move out of engagement with the nib such that the cap can be completely removed from the outer housing.
16. The medicament delivery device of any of clauses 11-15, further comprising:

a delivery member connected to a proximal end of a medicament container positioned within the outer housing;

a rigid delivery member shield surrounding and covering a distal end of the delivery member; and

a shield remover, wherein the distal portion further comprises a delivery device connector configured to releasably engage a corresponding connector located on the outer housing.
17. The medicament delivery device of clause 16, where the shield remover comprises:

a hollow tube that projects distally from the interior surface parallel to the longitudinal axis and extends beyond a circumferential distal end of the cap;

a grabber assembly configured to engage the rigid delivery member shield.
18. The medicament delivery device of clause 17, where the hollow tube comprises a first part of a grabber assembly configured to engage the rigid delivery member shield.
19. The medicament delivery device of clause 18, wherein the grabber assembly further comprises a second part comprising at least two distally extending fingers each terminating in a shield hook.
20. The medicament delivery device of clause 19, where the shield hooks engage the rigid delivery member shield when the cap is moved in a distal direction relative to the outer housing causing the rigid delivery member shield to disengage from the delivery member.
21. A medicament delivery device comprising:

a locking mechanism according to any of clauses 1 to 10; and

a medicament delivery member cover comprising a protrusion configured to engage with the second slot.

1-17. (canceled)

18. A locking mechanism for preventing axial movement of a medicament delivery member cover in a medicament delivery device, the locking mechanism comprising:
a cap with a longitudinal axis;
a locking ring having an annular shape and being rotatably positioned within an interior of the cap;
a medicament delivery member cover rotatably positioned within an interior of the locking ring;
a first connection between the cap and the locking ring, where the first connection comprising a first slot and a first protrusion in the first slot, where the first slot is in one of the locking ring and the cap and the first protrusion is attached to the other of the locking ring and the cap; and
a second connection between the locking ring and the medicament delivery member cover, where the second connection comprising a second slot and a second protrusion arranged in the second slot, where the second slot is in one of the locking ring and the medicament delivery member cover, and the second protrusion is attached to the other of the locking ring and the medicament delivery member cover.

19. The locking mechanism of claim 18, wherein the first protrusion is attached to the cap, and the first protrusion projects radially inward from an interior portion of the cap towards a longitudinal axis of the cap.

20. The locking mechanism of claim 18, wherein the second slot is in the locking ring, and the second slot is located at a distal end of the locking ring and has an open end.

21. The locking mechanism of claim 18, wherein the second slot is in the medicament delivery member cover, and the second slot is located at a proximal end of the locking ring and has an open end.

22. The locking mechanism of claim 20, wherein the second slot extends from the open end to a closed end, and wherein the second protrusion is in the second slot at the closed end.

23. The locking mechanism of claim 18, wherein the locking ring is configured to rotate relative to the cap from a locked position to an unlocked position when the cap is moved axially relative to the medicament delivery member cover.

24. The locking mechanism of claim 18, wherein when the locking ring is in the locked position the medicament delivery member cover cannot move axially relative to an outer housing of the medicament delivery device.

25. The locking mechanism of claim 18, wherein the first slot comprises a helical cam.

26. The locking mechanism of claim 25, wherein the helical cam comprises a cam surface against which the protrusion is pushed during use.

27. The locking mechanism of claim 18, where the second slot is an L-shaped cut-out.

28. The locking mechanism of claim 18, wherein the locking ring extends from a proximal end to a distal end relative to the longitudinal axis, and wherein the second slot is spaced apart from the proximal end and from the distal end in a direction relative to the longitudinal axis.

29. The locking mechanism of claim 18, where the first protrusion comprises a cam follower that is always slidably engaged with a cam surface on the first slot.

30. The locking mechanism of claim 18, wherein the cap further comprises a hollow tube that projects distally from the interior portion parallel to the longitudinal axis and extends beyond a circumferential distal end of the cap.

31. The locking mechanism of claim 30, wherein the tube comprises a first part of a grabber assembly configured to engage a rigid delivery member shield.

32. The locking mechanism of claim 31, where the grabber assembly further comprises a second part comprising at least two distally extending fingers, each terminating in a shield hook.

33. The locking mechanism of claim 18, wherein the first slot and the second slot both extend the same distance around the longitudinal axis.

34. A medicament delivery device comprising the locking mechanism of claim 18.

35. A locking mechanism for preventing axial movement of a medicament delivery member cover in a medicament delivery device, the locking mechanism comprising:
a cap with a longitudinal axis;
a locking ring positioned within an interior of the cap, having an annular shape and being rotatable relative to the cap from a locked position to an unlocked position when the cap is moved axially relative to the medicament delivery member cover;
a medicament delivery member cover rotatably positioned within an interior of the locking ring;
a first connection between the cap and the locking ring, where the first connection comprising a first slot and a first protrusion in the first slot, where the first slot is in one of the locking ring and the cap and the first protrusion is attached to the other of the locking ring and the cap; and
a second connection between the locking ring and the medicament delivery member cover, where the second connection comprising a second slot and a second protrusion arranged in the second slot, where the second slot is in one of the locking ring and the medicament delivery member cover, and the second protrusion is attached to the other of the locking ring and the medicament delivery member cover.

36. The locking mechanism of claim 35, wherein when the locking ring is in the locked position the medicament delivery member cover cannot move axially relative to an outer housing of the medicament delivery device.

37. The locking mechanism of claim 36, wherein the first slot comprises a helical cam having a cam surface against which the protrusion is pushed during use.
